(19) United States
(12) Patent Application Publication
(10) Pub. No.: US 2017/0312793 A1
(43) Pub. Date: Nov. 2, 2017

(54) UV-UVV CURABLE, WEAR-RESISTANT NAIL POLISH

(71) Applicant: Kegel, LLC, Lake Wales, FL (US)

(72) Inventor: Elena Y. Komarova, Stokesdale, NC (US)

(21) Appl. No.: 15/647,952

(22) Filed: Jul. 12, 2017

Related U.S. Application Data
(63) Continuation-in-part of application No. 14/496,770, filed on Sep. 25, 2014.
(60) Provisional application No. 61/882,400, filed on Sep. 25, 2013.

(51) Int. Cl. B08B 3/08; A61K 8/87; A61Q 3/02; A61K 8/02; A61K 8/81; A61K 8/86

(52) U.S. Cl. CPC B08B 3/08; A61K 8/8152; A61K 8/86; A61Q 3/02; A61K 8/0233; A61K 8/87; A61K 2800/43; A61K 2800/81; A61K 2800/30

(57) ABSTRACT

A novel nail polish system is provided. The system includes a non UV-curable base/adhesion coat, a UV- (or UV LED-) curable, water-removable color coat, and a UV- (or UV LED-) curable, highly wear-resistant top coat. The nail color includes water soluble components, making it removable by an aqueous remover once the top coat is breached, allowing water contact. The top coat composition comprises high concentrations of inorganic nanoparticles, thus providing superior scratch protection.

### RELATED APPLICATION

The present application is a continuation-in-part of U.S. patent application Ser. No. 14/496,770, filed Sep. 25, 2014, entitled UV-UVV CURABLE, WEAR-RESISTANT NAIL POLISH, and incorporated by reference in its entirety herein. U.S. patent application Ser. No. 14/496,770 claims the priority benefit of U.S. Provisional Patent Application Ser. No. 61/882,400, filed Sep. 25, 2013, entitled UV-UVV CURABLE, WEAR-RESISTANT NAIL POLISH, incorporated by reference in its entirety herein.

### BACKGROUND OF THE INVENTION

### Field of the Invention

The present invention relates to a novel, UV-UVV curable nail polish system with high durability, but that can also be removed with an aqueous remover.

### Description of the Prior Art

Nail garnishment dates back to ancient times. It existed in ancient China and Egypt. Nail polish as it is known today—a pigmented, solvent-borne polymer coating—started in the beginning of the 20th century, when pigmented automotive paint and nitrocellulose/cellulose acetate films were discovered. Nail polish formulations have changed very little since pigmented nitrocellulose, or its less flammable counterpart cellulose acetate, was used to polish nails, despite high flammability and adverse health effects of some of the nail polish components. There were attempts to make a waterborne nail polish, but these prior attempts suffered from low wear resistance and also required long drying times due to the slow water evaporation rate.

Only recently, a new turn in nail polish research found that UV curable polishes generally have improved scratch and wear resistance. Another advantage of these polishes is their shorter dry time. It takes only a couple of minutes under UV light to completely dry the polish. UV light in the nail drying machine has a wavelength of about 360-385 nm, which is the UV A portion of the spectrum. These types of rays are damaging to the skin if the intensity is high. The intensity of light of the nail UV lamps is very low, so the damage/risk that one gets from that exposure is very low as well. There have been some attempts to change to a safer, 395-nm wavelength of UV LED, which is closer to visible light. This wavelength is safer than light at 365 nm, but still presents some danger to skin. In any case it is better to minimize the exposure of skin to this type of light.

High energy and skin-damaging UV radiation such as UVC (100-290 nm) is required to effectively cure standard UV coatings that react through free radical mechanisms (e.g., such as acrylate/methacrylate coatings in ambient conditions). Ambient conditions here are defined as the presence of oxygen at concentrations that are equal to or above 20% by volume, which can be translated into 18 kPa oxygen partial pressure. In these conditions, most of the existing free-radical UV curable coatings will not sufficiently cure under UVA light, even if UVA absorbing initiators are applied. The surface of the coating does not completely cure, i.e., it remains tacky after UVA exposure due to oxygen inhibition. The practice of applying a nitrogen blanket in order to properly cure these UV coating compositions is customary even when mercury vapor UV lamps are utilized. This is especially true if high scratch- or abrasion-resistance is required. There are several antioxidants that can offset oxygen presence in the formulation to be cured. Examples of these include thiols, phosphites, amines, and borates. In UV nail polish, additives that prevent oxygen inhibition such as tertiary amines as well as multifunctional thiols have been applied to overcome this problem, as well as to improve dry-to-touch time. However, due to its acidic nature, thiols can cause skin irritation. In some prior art thiol-containing compositions, the thiol is present at concentrations of about 2%, which further means that the composition is likely to exhibit unwanted oxygen-induced polymerization in the bottle without irradiation with UV light.

### SUMMARY OF THE INVENTION

The present invention broadly provides a method of removing nail polish with water. This removal method comprises abrading the surface of cured nail polish on a nail. The cured nail polish is then contacted with water for a sufficient time to soften it, thus forming softened nail polish, and the softened nail polish is removed from the nail.

### DETAILED DESCRIPTION OF THE PREFERRED EMBODIMENTS

The present invention is broadly concerned with a nail polish system, comprising one or more of the following types of layers.

### Base Coat

Conventional base coats are suitable for use in the present invention. Preferred base coats are formed from functional silanes, and include those selected from the group consisting of pure aminosilanes, epoxy silanes, and hydroxysilanes. Other suitable silanes include those selected from the group consisting of silanes comprising unsaturated acrylic/methacrylic or vinyl ether functionalities. These can be obtained from Momentive, Dow Corning, and Al2Chem.

### Color Layer

The inventive color coating will typically be formed from a composition comprising one or more monomeric and/or oligomeric components, along with a color-imparting agent and other ingredients.

Preferred such monomeric/oligomeric components are water soluble or water-swellable (forming a hydrogel). As used herein, “water soluble” means that these components can at least substantially, and preferably completely, dissolve in water, or swell or become very soft upon absorbing water. Preferred such water soluble components comprise acrylates and methacrylates. Preferred methacrylates include those selected from the group consisting of hydroxyethyl methacrylate, hydroxypropyl methacrylate, polyethyleneglycole-modified methacrylates, isobornyl methacrylate, tetrahydrofurfuryl methacrylate, di-hydroxyethylmethacrylate trimethylhexyl dicarbamate, and aliphatic or aromatic polyether urethanes methacrylates.

These water soluble components will preferably have a weight average molecular weight of less than about 30,000 Daltons, preferably less than about 25,000 Daltons, and even more preferably from about 300 Daltons to about 25,000 Daltons. These components will typically be included in the composition at levels of from about 30% by weight to about 99% by weight, preferably from about 40% by weight to about 98% by weight, and even more preferably from about 50% by weight to about 80% by weight, based upon the total weight of the color layer composition taken as 100% by weight.

The preferred color layer composition can also comprise non-crosslinkable (not having an unsaturated bond capable of participating in radical polymerization), water soluble components such as a modified polyether ketone resin (such as VariPlus UC, available from TEGO Evonik) or an acetone soluble polyol resin (such as VariPlus SK, available from TEGO Evonik). These component may be included separately or together in the color layer composition. When included, the total levels of all such components is from about 1% by weight to about 40% by weight, more preferably from 5% by weight to about 30% by weight, and even more preferably from about 5% by weight to about 25% by weight, based upon the total weight of the color layer composition taken as 100% by weight.

The color-imparting agents used in the present invention include those selected from the group consisting of pigments, dyes, special effect pigments, optical brighteners, and other fluorescent and coloring agents. These agents will typically be present at high levels in order to reach the desired color density in fewer layers of the coating, thus minimizing exposure to UV light. These levels are preferably from about 0.1% by weight to about 40% by weight, preferably from about 1% by weight to about 30% by weight, and even more preferably from about 5% by weight to about 25% by weight, based upon the total weight of the color layer composition taken as 100% by weight.

The color layer compositions will also comprise a photoinitiator suitable for the intended wavelength of use. Preferred photoinitiators are phosphine oxide photoinitiators, with particularly preferred photoinitiators including those selected from the group consisting of bis acyl phosphine oxide (BAPO), diphenyl(2 4 6-trimethylbenzoyl)phosphine oxide, and ethyl (2,4,6-trimethylbenzoyl) phenylphosphinate. The photoinitiator is preferably present at levels of from about 0.1% by weight to about 40% by weight, preferably from about 0.5% by weight to about 10% by weight, and even more preferably from about 1% by weight to about 5% by weight, based upon the total weight of the color layer composition taken as 100% by weight.

Preferred color layer compositions will also preferably comprise surface energy altering components (i.e., leveling, antifoaming, and/or flow additives) in order to reduce the surface energy of the composition. Examples of preferred surface energy altering components include those selected from the group consisting of siloxane surfactants, fluorosurfactants, and acrylic surfactants. When utilized, the surface energy altering component is preferably present at levels from about 0.01% by weight to about 6% by weight, and preferably from about 0.01% by weight to about 3% by weight, based upon the total weight of the color layer composition taken as 100% by weight.

The color layer compositions utilized in the invention can also include polymeric additives, depending upon the desired properties and other components utilized. Such additives include viscosity-adjusting polymers, gloss-improving polymers, and hardening polymers. These types of additives are utilized at levels of less than about 60% by weight, preferably from about 0.1% by weight to about 45% by weight, and even more preferably from about 0.1% by weight to about 30% by weight, based upon the total weight of the color layer composition taken as 100% by weight.

Finally, the color layer compositions might include a solvent, depending upon the embodiment. Examples of suitable solvents include those selected from the group consisting of acetone, n-butyl actetae, ethyl actetate, tert-butyl acetate, and toluene. In embodiments where a solvent is included, it is preferably present at levels of from about 0.1% by weight to about 30% by weight, and preferably from about 0.1% to about 25% by weight, based upon the total weight of the color layer composition taken as 100% by weight.

In one embodiment, the color layer comprises a VOC-exempt solvent. As used herein, a VOC solvent means one of carbon (excluding carbon monoxide, carbon dioxide, carbonic acid, metallic carbides or carbonates, and ammonium carbonate) that participates in atmospheric photochemical reactions. VOC-exempt solvents are those that have negligible or no photochemical activity and are defined in 40 C.F.R. 51.100(s)(1), incorporated by reference herein. Preferred VOC-exempt solvents for use in the present invention include those selected from the group consisting of dimethyl carbonate, acetone, methyl acetate, p-chlorobenzotrifluori de, propylene carbonate, t-butyl acetate, and mixtures thereof. When a VOC-exempt solvent is utilized, it is preferably present at the levels discussed above with respect to solvents generally.

In another embodiment, the color layer composition is essentially free of any solvents. In other words, the composition would comprise less than about 0.01% by weight solvent, and preferably about 0% by weight solvent, based upon the total weight of the color layer composition taken as 100% by weight.

### Top Coat

The top coat will typically be formed from a composition comprising one or more monomeric and/or oligomeric components, along with a filler and other components.

Preferred such monomeric/oligomeric components comprise radical-curing monomers and oligomer selected from the group consisting of acrylates, methacrylate, and epoxies. Particularly preferred such components are selected from the group consisting of aliphatic urethane acrylates, epoxy acrylates, melamine acrylates, and/or multifunctional acrylates as well as di- and monofunctional acrylates. Also, other ethylenically unsaturated monomers that can be polymerized via radical initiation route and are derived from vinyl, allyl, methallyl esters ether and amines are suitable radical-curing monomers are suitable.

Further preferred such components for use in the top coat are selected from the group consisting of the following acrylates: trimethylol propane triacrylate (TMPTA), 1,6-hexanediol acrylate, isobornyl acrylate, hexafunctional urethane acrylate, hexafunctional epoxy acrylate, tripropylene glycol diacrylate, epoxy novolak acrylate, ethoxylated trimethylolpropane triacrylate, dipentaerythritol pentaacrylate, and/or many others. Preferred methacrylates include those selected from the group consisting of polyethyleneglycole-modified methacrylates, isobornyl methacrylate, tetrahydrofurfuryl methacrylate, di-hydroxyethylmethacrylate trimethylhexyl dicarbamate, aliphatic or aromatic polyether urethanes methacrylates, and/or dimethacrylates, available from companies such as GEO Specialty Chemicals, UK.

These monomers and oligomers will preferably have a weight average molecular weight of less than about 30,000 Daltons, preferably less than about 25,000 Daltons, and even more preferably from about 300 Daltons to about 25,000 Daltons. These components will typically be included in the composition at levels of from about 15% by weight to about 85% by weight, preferably from about 20% by weight to about 80% by weight, and even more preferably from about 30% by weight to about 70% by weight, based upon the total weight of the top coat composition taken as 100% by weight.

The fillers included in the compositions used to form the inventive top coat can be either an organic or an inorganic filler particles or nanoparticles. These filler particles or nanoparticles will be dispersed through and among the monomeric/oligomeric components and provide abrasion resistance and hardness in the top coating.

The filler is preferably provided at higher levels of at least about 5% by weight, preferably at least about 15% by weight, more preferably at least about 20% by weight, and even more preferably from about 30% by weight to about 60% by weight, based upon the total weight of the top coat composition taken as 100% by weight. In some embodiments, the inventive composition is essentially free of any fillers (such as nanoparticles). That is, in these embodiments, the compositions comprise less than about 0.1% by weight, preferably less than about 0.01% by weight, and preferably about 0% by weight fillers, based upon the total weight of the top coat composition or cured top coat taken as 100% by weight.

Preferred nanoparticles comprise alumina and/or silica nanoparticles. Particularly preferred such nanoparticles are available from Nanoresins (Geesthacht, Germany; now a part of Evonik), Nanophase Technologies (Romeoville, Ill.), Nissan Chemical America Corporation (Houston, Tex.), and Eternal Chemical Ltd (China). Other suitable filler particles are selected from the group consisting of siloxane resin particles, such as Albidur line of dispersions available from Evonik Tego.

The top coat compositions will also comprise a radical photoinitiator and/or sensitizers suitable for the intended wavelength of use (preferably from about 360 nm to about 420 nm). Preferred photoinitiators are phosphine oxide photoinitiators. Typical photoinitiators include those selected from the group consisting of bis acyl phosphine oxide (BAPO), (e.g., Irgacure 819, Irgacure 819 DW, available BASF), diphenyl(2 4 6-trimethylbenzoyl)phosphine oxide (Chivacure TPO, available from Chitec Technology Corp., Ltd), and ethyl (2,4,6-trimethylbenzoyl) phenylphosphinate (Speedcure TPO-L, available from Lambson Limited, West Yorkshire, UK), while typical photosensitizes are selected from the group consisting of diethylthioxanothone and ispropylthioxanothone photosensitizers. The photoinitiator and photo sensitizer are preferably each present at levels of from about 0.5% by weight to about 15% by weight, preferably from about 1% by weight to about 10% by weight, and even more preferably from about 1% by weight to about 8% by weight, based upon the total weight of the top coat composition taken as 100% by weight.

Preferred top coat compositions will also preferably comprise surface energy altering components (i.e., leveling, antifoaming, and/or flow additives) in order to reduce the surface energy of the composition. Examples of preferred surface energy altering components include those selected from the group consisting of siloxane surfactants, fluorosurfactants, and acrylic surfactants. These additives are available from BYK-Chemie GmbH (Wesel, Germany), Evonik Tego Chemie GmbH (Essen, Germany), and 3M (St. Paul, Minn.), for example. Many of the additives used have an embedded acrylate functionality. When utilized, the surface energy altering component is preferably present at levels from about 0.01% by weight to about 6% by weight, and preferably from about 0.01% by weight to about 3% by weight, based upon the total weight of the top coat composition taken as 100% by weight.

The top coat compositions utilized in the invention can also include polymeric additives, depending upon the desired properties and other components utilized. Such additives include viscosity-adjusting polymers, gloss-improving polymers, hardening polymers, and visoscity-adjusting polymers. Examples of preferred polymeric additives include those selected from the group consisting of modified polyether ketone resin, polyol resin, polyacrylate resins, and polymethacrylate resins.

These types of additives are utilized at levels of less than about 45% by weight, preferably from about 0.1% by weight to about 40% by weight, and even more preferably from about 0.1% by weight to about 30% by weight, based upon the total weight of the top coat composition taken as 100% by weight.

Finally, the top coat compositions may include a solvent, depending upon the embodiment. Examples of suitable solvents include those selected from the group consisting of acetone, n-butyl actetae, ethyl actetate, tert-butyl acetate, and toluene. In embodiments where a solvent is included, it is preferably present at levels of from about 0.1% by weight to about 30% by weight, and preferably from about 0.1% to about 25% by weight.

In one embodiment, the top coat composition comprises a VOC-exempt solvent. Preferred VOC-exempt solvents for use in the inventive top coat include those selected from the group consisting of dimethyl carbonate, acetone, methyl acetate, p-chlorobenzotrifluoride, propylene carbonate, t-butyl acetate, and mixtures thereof. When a VOC-exempt solvent is utilized, it is preferably present at the levels discussed above with respect to solvents generally present in the top coat.

In one embodiment, the top coat layer composition is essentially free of any solvents. In other words, the top coat composition would comprise less than about 0.01% by weight solvent, and preferably about 0% by weight solvent, based upon the total weight of the composition taken as 100% by weight.

### Method of Using the Nail Compositions

The method of using the nail composition preferably involves first applying the base coat to the nail surface. It can be wiped with a cloth soaked in the base coat, sprayed on the nail, or applied by any other suitable means of forming a layer of the base coat on the nail. The nail surface is then preferably wiped with a dry cloth in order to make the layer of silane thinner. The application of this base coat does not require UV light to cure, and does not require prolonged drying time or any drying time at all. Rather, the nail surface is ready for the color layer application immediately after this adhesion layer or base layer application. The base coat provides exceptional adhesion to different substrates including human nails, artificial nails, and nail extensions.

Next, the color layer is formed on the base coat. As with the base coat, it can be applied by any means capable of forming a smooth layer on the nail, with brushing being the most preferred. The layer is then cured by exposure to UV or UV LED light at the desired wavelength. Advantageously, any commercially available nail lamp is suitable for use with this invention. This curing would typically be accomplished at a wavelength of from about 300 nm to about 800 nm, and preferably from about 350 nm to about 600 nm. Typical curing times are from about 10 seconds to about 180 seconds, and more preferably from about 15 seconds to about 120 seconds. The cured color layer is preferably water swellable, and is also preferably a thin layer, with a typical average thickness (an average taken over 5 measurements) being less than about 0.004 inch, and preferably from about 0.002 inch to about 0.003 inch. In a preferred embodiment, at least one more color layer of the above thickness is formed, followed by the above curing conditions.

Finally, the inventive top coat is applied to the cured color layer. Again, this can be applied by any means capable of forming a smooth protective layer on the color layer, with brushing being the most preferred. The layer is then cured by exposure to UV or UV LED light at the desired wavelength. Again, any commercially available nail lamp is suitable for curing this top coat layer. Curing of the top coat would typically be accomplished at a wavelength of from about 300 nm to about 800 nm, and preferably from about 350 nm to about 600 nm. Typical curing times are from about 20 seconds to about 200 seconds, and more preferably from about 25 seconds to about 180 seconds. The cured top coat is preferably a thin layer, with a typical average thickness (an average taken over 5 measurements) being less than about 0.005 inch, and preferably from about 0.002 inch to about 0.003 inch. If desired, at least one more top coat layer of the above thickness can be formed, following the above curing conditions.

Because the top coat comprises from about 20% to about 60% by weight of inorganic or organic particles, the final nail polish system is highly scratch- and abrasion-resistant. For example, when subjected to an abrasion test, the cured top coat will have a ΔHaze of less than about 30, preferably less than about 20, and more preferably from about 0 to about 10. The entire coating system (i.e., base+color+top coats) will also have an adhesion to a natural, acrylic, or other synthetic nail of at least about 90%, preferably at least about 95%, and more preferably about 100%.

As used herein, “scratch” is tested with a 000 steel wool, 20 rubs, with no weight applied. The results are inspected visually. “Abrasion” is tested with a TABER® Rotary Platform Abrasion Tester. The coating is subjected to 100 cycles with a 500-gram load and CS-10F wheels. The ΔHaze of a coating is determined before and after the abrasion, and the change in haze is recorded. The test is performed on a glass slide or bi-axially oriented polyethylene terephthalate film.

“Adhesion” is determined by scribing the surface of the nail polish with a razor in the shape of an X. Scotch tape (available from 3M) is attached to the surface of the nail polish over the X with pressure from a rubber eraser. A tab of the tape is left detached from the surface for grasping with fingers. After 2 minutes, the tape is removed rapidly and the percentage of the area where the tape is attached is assessed to determine the percent remaining.

Additional benefits of the high filler content is that the resulting coating provides a superior water barrier. That is, the nail system could be soaked in water for a time period of at least about 30 minutes, and even from about 60 minutes to about 120 minutes, without softening or compromising the layer (i.e., the system will still have properties within the ranges defined above). In embodiments where there are nanoparticles dispersed within the color layer, pigment sedimentation is slowed down, making pigmented dispersions more stable.

In spite of the above properties, the present invention provides a significant advantage over prior art UV-curable nail systems in that it can be removed with water or other aqueous solutions, in addition to any conventional nail polish removal solutions (e.g., acetone). The removal process (regardless of the remover utilized) involves first compromising the top coat layer by abrading with a rough surface, such as a nail file. This allows the remover solution to penetrate past the top coat and into the underlying layers. Next, the nail system is soaked in the remover solution at ambient conditions for a time period of from about 2 minutes to about 30 minutes, and preferably from about 5 minutes to about 10 minutes, during which time the color layer is softened by the solution. The softened color layer can then be peeled from the nail.

Because the inventive removal process involves using an aqueous solutions, it will be appreciated that harsh chemicals can be avoided during the removal process, making a highly durable nail color system that stays in place until removal is desired, with that removal being a less burdensome process than prior art removal processes. Examples of harsh chemicals that can be avoided include those selected from the group consisting of acetone, acetonitrile, ethyl acetate, butyl acetate, methyl ethyl ketone, toluene, and isopropyl alcohol. In a preferred embodiment, the aqueous remover solution comprises less than about 3%, preferably less than about 1%, and more preferably about 0% by weight total of these harsh chemicals, based upon the total weight of the aqueous remover solution taken as 100% by weight. In a particularly preferred embodiment, the aqueous remover solution comprises less than about 1% by weight acetone, and preferably about 0% by weight acetone, based upon the total weight of the aqueous remover solution taken as 100% by weight.

In another embodiment, the aqueous remover solution comprises at least about 90% water, preferably at least about 95% water, and more preferably about 100% water, based upon the total weight of the aqueous remover solution taken as 100% by weight. In a further embodiment, the aqueous remover solution consists essentially of, or even consists of, water. In those embodiments where the aqueous remover solution is not 100% water, the balance will not include the foregoing harsh chemicals, but will instead be various optional ingredients such as conditioners, surfactants, emollients, fragrances, coloring agents, vitamins, ph adjustors, buffers, and combinations of the foregoing. The foregoing remover options apply to both colored polishes and colorless or transparent polishes.

### EXAMPLES

The following examples set forth preferred methods in accordance with the invention. It is to be understood, however, that these examples are provided by way of illustration and nothing therein should be taken as a limitation upon the overall scope of the invention.

### Materials and Methods

Components of the coating composition were mixed in ambient conditions with light exposure kept to a minimum. Plastic (polyethylene) containers were used for mixing with a laboratory mixer equipped with a stainless steel blade. Silica nanoparticle dispersions in acrylic monomers (Nanocryl) were received as samples from Nanophase Technologies (Romeoville, Ill., now part of Evonik Tego Chemie GmbH, Essen, Germany). Other monomers were received as samples from either Sartomer USA, LLC (Exton, Pa.) or Cytec Industries (Woodland Park, N.J.). Methacrylate monomers/oligomers under the brand name of Bisomer were received as samples 30 from GEO Specialty Chemicals UK Ltd (Hampshire, UK). Other methacrylate monomers/oligomers was obtained from RAHN USA Corp. (Aurora, Ill.), Rad Solutions LLC (Flower Mound, Tex.). Photoinitiators were samples from BASF or Aalchem (Grand Rapids, Mich.).

The coatings were applied to nail or artificial nail surfaces with a regular nail polish brush. The coatings were cured with a UV light source: professional UV gel nail dryer by “Royal Nails,” model RN 541, as well as with a UV LED light source: 4 W LED nail lamp (Gellish, Hand&Nail Harmony, Brea, Calif.). In the case of the UV unit, the pigmented coating was dried for 2 minutes at 365 nm, and the top coat was dried for 3 minutes at 365 nm. In case of UV LED unit, the color coat was dried for 45 seconds at 395 nm, and the top coat was dried for 1.5 minutes at 395 nm. No UV cure was utilized for base/adhesion coat. The sticky layer was removed by wiping the surface of the top coat with isopropyl alcohol (rubbing alcohol).

### Example 1

### Base Coat

The base coat was 3-glycidoxypropyltrimethoxysilane (Al 160 epoxy silane, available from Al2Chem, New Jersey, USA). A cotton ball soaked in the silane was used to apply the base coat, and a paper towel was used to wipe away the excess from the nail.

### Example 2

### Top Coat

Various top coat formulations were prepared, as shown in Tables 2.1-2.4.

TABLE 2.1

| COMPONENT | WEIGHT % |
|---|---|
| Nanocryl C-153 | 90 |
| Sartomer CD9053 | 4.9 |
| Irgacure 819 | 5 |
| Effka 3883 | 0.1 |

TABLE 2.2

| COMPONENT | WEIGHT % |
|---|---|
| Nanocryl C-153 | 88 |
| Albidur 5340 | 2 |
| Sartomer CD9053 | 4.8 |
| Irgacure 819 | 5 |
| TEGO FLOW 370 | 0.18 |
| FC 4430 | 0.02 |

TABLE 2.3

| COMPONENT | WEIGHT % |
|---|---|
| Nanocryl C-145 | 92 |
| VariPlus SK | 6.49 |
| P1920 | 1 |
| TEGO Flow 370 | 0.5 |
| FC3340 | 0.01 |

TABLE 2.4

| COMPONENT | WEIGHT % |
|---|---|
| Nanocryl C-145 | 68.8 |
| VariPlus SK | 8.8 |
| PI 920 | 1 |
| TEGO Disperse 688 | 2.6 |
| BYK 1790 | 0.13 |
| BYK 399 | 0.3 |
| TEGO Rad 2250 | 0.07 |
| Acetone | 9.2 |
| Citraflex | 9.1 |

TABLE 2.5

| COMPONENT | WEIGHT % |
|---|---|
| Rahn UA-09-293 (aliphatic urethane acrylate resin, from | 60 |
| Rahn USA Corp., Aurora, IL) | |
| Radsol ABI (mixture of monomers, from Rad Solutions | 18 |
| LLC, Flower Mound, TX) | |
| TPO-L | 1.5 |
| BYK 1790 (polyacrylate-based surface additive, from | 0.3 |
| BYK-Chemie GmbH, Wesel, Germany) | |
| RAD 2100 (silicone acrylate surfactant, from Evonik | 0.2 |
| Tego Chemie GmbH, Essen, Germany) | |
| CAB 381-0.5 (25% in TergoSol) (cellulose acetate butyrate | 15 |
| ester, from Eastman Chemical, Kingsport, TN) | |
| n-Butyl Acetate | 5 |

### Example 3

### Color Coat

Various color coat formulations were prepared, as shown in Tables 3.1-3.7.

TABLE 3.1

| COMPONENT | WEIGHT % |
|---|---|
| Bisomer HEMA | 86 |
| Bisomer PEGDMA | 10 |
| Irgacure 819 | 3.8 |
| TEGO Flow 370 | 0.2 |

TABLE 3.2

| COMPONENT | WEIGHT % |
|---|---|
| TEGDMA | 45 |
| VariPlus SK | 20 |
| CD9054 | 4 |
| Irg 819 | 2 |
| TEGO Flow 370 | 0.4 |
| Xirona Volcanic Fire | 11 |
| Pigment | |
| Phthalo Blue R | 17.6 |
| UCD4830SF | |

TABLE 13

| COMPONENT | WEIGHT % |
|---|---|
| PEG₂₀₀ DMA | 45 |
| VariPlus SK | 20 |
| CD9054 | 4 |
| Irg 819 | 2 |
| TEGO Flow 370 | 0.4 |
| Xirona Volcanic Fire | 11 |
| Pigment | |
| Quinacridone Red, | 17.6 |
| UCD7959SF | |

TABLE 3.4

| COMPONENT | WEIGHT % |
|---|---|
| Irgacure 819 | 2 |
| Rahn 11-704 | 54 |
| Quinacridone Red, | 17 |
| UCD7959SF | |
| VariPlus UC | 10 |
| Acetone | 17 |

TABLE 3.5

| COMPONENT | WEIGHT % |
|---|---|
| Rahn 11-704 | 40 |
| Irgacure 819 | 2 |
| Quinacridone Red, | 18 |
| UCD7959SF | |
| VariPlus UC | 8 |
| VariPlus SK | 14 |
| Acetone | 18 |

TABLE 3.6

| COMPONENT | WEIGHT % |
|---|---|
| Rahn 11-704 | 40 |
| Irgacure 819 | 2 |
| Quinacridone Red, | 18 |
| UCD7959SF | |
| VariPlus UC | 8 |
| VariPlus AP | 14 |
| Acetone | 18 |

TABLE 3.7

| COMPONENT | WEIGHT % |
|---|---|
| Rahn 11-704 | 33 |
| VariPlus SK | 22 |
| Acetone | 18 |
| Quinacridone Red, | 15 |
| UCD7959SF | |
| Irgacure 819 | 1.8 |
| VariPlus UC | 6 |
| TEGO Disperse 688 | 2 |
| BYK 1790 | 0.2 |
| Xirona Volcanic Fire | 2 |
| Pigment | |

TABLE 3.8

| COMPONENT | WEIGHT % |
|---|---|
| IPUDMA-TD10 (mixture of monomers and oligomers, from | 20 |
| Rad Solutions LLC, Flower Mound, TX) | |
| Rahn 11-704 | 20 |
| VariPlus 1201-TF (solution of a polyurethane polyol resin, | 8.6 |
| from Evonik Tego Chemie GmbH, Essen, Germany) | |
| VP-D-354 (wetting and dispersing material, from Evonik | 1 |
| Tego Chemie GmbH, Essen, Germany) | |
| BYK 1790 | 0.3 |
| BYK 361N | 1.5 |
| Phthalo Blue R UCD4830SF | 12.6 |
| Xirona Purple Sparkle | 1.5 |
| TPO-L | 1.5 |
| SR9051 (an adhesion methacrylic acid promoter resin, from | 5 |
| Sartomer USA, LLC, Exton, PA) | |
| VariPlus SK (50% in TergoSol) | 26 |
| Aerosil 380 (hydrophilic fumed silica with a specific surface | 2 |
| area of 380 m²/g, from Evonik Tego Chemie GmbH, Essen, | |
| Germany) | |

TABLE 3.9

| COMPONENT | WEIGHT % |
|---|---|
| IPUDMA-TD10 | 24 |
| Rahn 11-704 | 24 |
| VariPlus 1201-TF | 9 |
| BYK 1790 | 0.5 |
| BYK 361N | 1.7 |
| TPO-L | 1.8 |
| SR9051 | 7 |
| VariPlus SK (50% in TergoSol) | 30 |
| Aerosil 380 | 2 |

### Example 4

### Removal

### 1. Aqueous Removal

Advantageously, the color layer can be removed by water or other aqueous solution. For aqueous removal, the top coat was abraded with a rough nail file in order to assist with water penetration through the top layer and into the color layer. After abrading, the nail was soaked in warm water for several minutes, which softened the color layer. Once softened, the polish was peeled off the nail.

### 2. Other Removal Solutions

In some instances, a typical nail polish remover (acetone) was utilized instead of water. The removal procedure was similar to that for aqueous removal. That is, the top layer was roughened by a file, the nails were then soaked in the remover for several minutes, and the polish was peeled off the nail after the color layer had softened.

The removal media depends entirely on the composition of the color coat. Table 4 shows the results of removal tests of the Example 3 color coats. In each instance, the top coat (any) was roughened with a coarse file, the nails were soaked for 10 minutes in acetone or water (as shown in Table 4), and the polish was then peeled from the nail.

TABLE 4

| COLOR COAT (2 | | |
|---|---|---|
| LAYERS) + TOP COAT | ACETONE | WATER |
| Example 3.1 | − | + |
| Example 3.2 | − | + |
| Example 3.3 | − | + |
| Example 3.4 | − | + |
| Example 3.5 | − | + |
| Example 3.6 | + | + |
| Example 3.7 | + | + |
| Example 3.8 | + | + |
| Example 3.9 | + | + |

+ could be removed after soaking in this solvent
− could not be removed after soaking in this solvent

### 3. Other Options for Aqueous Removal

Addition of acids, bases, and/or mineral or essential oils does not influence the nail polish removal process. The aqueous removal such as the one described above in Part 1 of this Example 4 can be done with moisturizing hand soaks containing olive, coconut oils. or similar natural or artificial moisturizer-containing water. The aqueous removal was successfully performed with hot water that contained essential oils such as lavender, almond, etc. Aqueous solutions including vinegar, orange juice, lime/lemon juice, and/or baking soda soak off the inventive nail polish efficiently as well.

### 4. Aqueous Removal Option

The aqueous removal such as the one described in Part 1 of this Example 4 can alternatively be performed without roughening or abrading the nail surface with a file. If one applies hot water (from about 100 to about 110° F.), the roughening of the nail surface is not necessary. The nail polish softens and can be peeled after 2-5 minutes of soaking in water having this temperature.

We claim:

1. A method of removing cured nail polish from a nail, said method comprising the steps of:
contacting the surface of cured nail polish on the nail with water for sufficient time to soften said cured nail polish and form softened nail polish; and
removing said softened nail polish from the nail.

2. The method of claim 1, further comprising abrading the surface of cured nail polish prior to said contacting with water.

3. The method of claim 2, wherein said cured nail polish comprises a UV- or UVV-cured top coat layer and a UV- or UVV-cured color coat layer under the top coat layer.

4. The method of claim 3, wherein said abrading comprises abrading said top coat layer.

5. The method of claim 3, wherein:
said cured color layer is formed from a composition comprising a water-soluble component, a photoinitiator, and a color-imparting agent; and
said cured top coat layer is formed from a composition comprising a monomeric and/or oligomeric component, a photoinitiator, and at least about 20% by weight filler.

6. The method of claim 3, wherein said cured top coat is essentially free of fillers.

7. The method of claim 1, wherein said contacting comprises soaking said cured nail polish in water for a time period of from about 2 minutes to about 30 minutes.

8. The method of claim 7, wherein said contacting comprises soaking said cured nail polish in water for a time period of from about 5 minutes to about 10 minutes.

9. The method of claim 1, wherein said removing comprises peeling said softened layer from the nail.

10. The method of claim 1, wherein harsh chemicals are avoided during said removing.

11. The method of claim 10, wherein acetone is avoided during said removing.

12. The method of claim 2, wherein prior to said abrading, said cured nail polish would not soften or be compromised if soaked in water for a time period of at least about 30 minutes.

13. The method of claim 2, wherein prior to said abrading, said cured nail polish would not soften or be compromised if soaked in water for a time period of from about 60 minutes to about 120 minutes.

14. A method of removing UV- or UV-cured nail polish from a nail, said method comprising the steps of:
contacting the surface of UV- or UV-cured nail polish on the nail with an aqueous remover solution for sufficient time to soften said cured nail polish and form softened nail polish, said aqueous remover solution comprising less than about 3% by weight of harsh chemicals selected from the group consisting of acetone, acetonitrile, ethyl acetate, butyl acetate, methyl ethyl ketone, toluene, and isopropyl alcohol, based upon the total weight of the aqueous remover solution taken as 100% by weight; and
removing said softened nail polish from the nail.

15. The method of claim 14, further comprising abrading the surface of cured nail polish prior to said contacting with said aqueous remover solution.

16. The method of claim 14, wherein said contacting comprises soaking said cured nail polish in said aqueous remover solution for a time period of from about 2 minutes to about 30 minutes.

17. The method of claim 16, wherein said contacting comprises soaking said cured nail polish in water for a time period of from about 5 minutes to about 10 minutes.

18. The method of claim 14, wherein said removing comprises peeling said softened layer from the nail.

19. A method of removing cured nail polish from a nail, said method comprising the steps of:
contacting the surface of cured nail polish on the nail with an aqueous remover solution for sufficient time to soften said cured nail polish and form softened nail polish, said aqueous remover solution comprising at least about 90% by weight of water, based upon the total weight of the aqueous remover solution taken as 100% by weight; and
removing said softened nail polish from the nail.

20. The method of claim 19, further comprising abrading the surface of cured nail polish prior to said contacting with said aqueous remover solution.

21. The method of claim 20, wherein said cured nail polish comprises a UV- or UVV-cured top coat layer and a UV- or UVV-cured color coat layer under the top coat layer.

22. The method of claim 21, wherein said cured top coat is essentially free of fillers.

23. The method of claim 19, wherein said contacting comprises soaking said cured nail polish in said aqueous remover solution for a time period of from about 2 minutes to about 30 minutes.

24. The method of claim 19, wherein said aqueous remover solution consists essentially of water.

25. The method of claim 19, wherein said removing comprises peeling said softened layer from the nail.
